(19) United States
(12) Patent Application Publication
(10) Pub. No.: US 2026/0348971 A1
(43) Pub. Date: Nov. 5, 2026

(54) METHOD FOR GENERATING NEGATIVE SAMPLES BASED ON RELATIONSHIP BETWEEN PEPTIDES AND SOURCE PROTEINS

(71) Applicant: Acer Incorporated, New Taipei City (TW)

(72) Inventors: Chih-Wei Tu, New Taipei City (TW); Yun-Hsuan Chan, New Taipei City (TW); Tsung-Hsien Tsai, New Taipei City (TW)

(73) Assignee: Acer Incorporated, New Taipei City (TW)

(21) Appl. No.: 19/251,747

(22) Filed: Jun. 26, 2025

(30) Foreign Application Priority Data
May 5, 2025 (TW) 114116728

(51) Int. Cl. G01N 33/68

(52) U.S. Cl. CPC G01N 33/6845; G01N 33/6878

(57) ABSTRACT

The invention provides a method for generating negative samples based on a relationship between peptides and source proteins, including the following steps. A source protein is marked, and global characteristics and regional characteristics of a protein sequence are defined via a relationship between a peptide and the protein sequence. Next, regional characteristic indicators and global characteristic indicators are calculated. Next, high-quality peptide mapping regions are screened out and negative samples are found in internal peptide mapping regions using the regional characteristic indicators, and high-quality protein sequences are screened out and negative samples are found in external peptide mapping regions using the global characteristic indicators.

### CROSS-REFERENCE TO RELATED APPLICATION

This application claims the priority benefit of Taiwan application serial no. 114116728, filed on May 5, 2025. The entirety of the above-mentioned patent application is hereby incorporated by reference herein and made a part of this specification.

### BACKGROUND OF THE INVENTION

### Field of the Invention

The invention relates to a method for generating negative samples, and more particularly to a method for generating negative samples based on the relationship between peptides and source proteins.

### Description of Related Art

The design of personalized mRNA cancer vaccines includes: related aspects such as prediction of tumor neoantigens, selection of peptide linkers, and mRNA sequence combinations, wherein the object of designing peptide linkers is to allow the mRNA vaccine to be accurately cleaved by protease after being injected into the human body. Currently, the design of peptide linkers is obtained by sorting the predicted probabilities of the protease cleavage prediction model and selecting the conditions. Therefore, the quality of the protease cleavage model indirectly affects the design of peptide linkers. During the training process of the protease cleavage model, since all training data are positive samples, negative samples need to be self-generated. Therefore, the object of the invention is to design a process for generating negative samples by integrating a plurality of perspectives to enhance the performance of the protease cleavage prediction model, which has the opportunity to further optimize the design of peptide linkers.

### SUMMARY OF THE INVENTION

The invention provides a method for generating negative samples based on the relationship between peptides and source proteins, so as to further optimize the design of peptide linkers.

A method for generating negative samples based on a relationship between peptides and source proteins of the invention includes the following steps. A source protein is marked, and global characteristics and regional characteristics of a protein sequence are defined via a relationship between a peptide and the protein sequence. Next, regional characteristic indicators and global characteristic indicators are calculated. Next, high-quality peptide mapping regions are screened out and negative samples are found in internal peptide mapping regions using the regional characteristic indicators, and high-quality protein sequences are screened out and negative samples are found in external peptide mapping regions using the global characteristic indicators.

In an embodiment of the invention, the global characteristic indicators include a cut point saturation, a peptide coverage, and a peptide density.

In an embodiment of the invention, the peptide density, the peptide coverage, and the cut point saturation are sorted from large to small to quantify a confidence level of a region without peptide coverage in the protein sequence as a negative sample.

In an embodiment of the invention, the regional characteristic indicators include defining high-frequency peptide mapping regions and calculating and sorting peptide mapping region quantitative indicators.

In an embodiment of the invention, defining the high-frequency peptide mapping regions includes selecting amino acid sites for which coverage times are greater than a median of coverage times of an entire amino acid site as qualified amino acid sites, and continuous amino acid sites form a peptide mapping region.

In an embodiment of the invention, the peptide mapping region quantitative indicators are calculated and sorted by calculating a peptide density, a peptide coverage, and a cut point saturation in units of peptide mapping regions.

In an embodiment of the invention, the peptide density, the peptide coverage, and the cut point saturation are sorted from large to small to quantify a confidence level of a region without peptide coverage in the protein sequence as a negative sample.

Based on the above, the invention provides a method for generating negative samples based on the relationship between peptides and source proteins. From the distribution relationship between peptides and protein sequences, regions with high frequency and high density of peptide distribution are found, and negative samples are defined therefrom. The characteristics of the source protein are described from a plurality of angles such as cut point and peptide distribution. Further, negative samples are found from regions without peptide coverage in the screened protein sequence, in order to find more representative negative samples. Therefore, the performance of the protease cleavage prediction model may be enhanced to further optimize the design of peptide linkers.

### BRIEF DESCRIPTION OF THE DRAWINGS

FIG. 1 is a schematic diagram of a conventional method for generating negative samples.

FIG. 2 is a schematic diagram for defining an internal peptide mapping region and an external peptide mapping region.

FIG. 3 is a schematic diagram of a method for generating negative samples based on the relationship between peptides and source proteins according to an embodiment of the invention.

FIG. 4 is a schematic diagram of finding negative samples in a defined region according to an embodiment of the invention.

### DESCRIPTION OF THE EMBODIMENTS

Embodiments are provided hereinafter and described in detail with reference to figures. However, the embodiments provided are not intended to limit the scope of the disclosure. In addition, the terms “contain”, “include”, “have”, etc., used in the article are all open terms, meaning “containing but not limited to”.

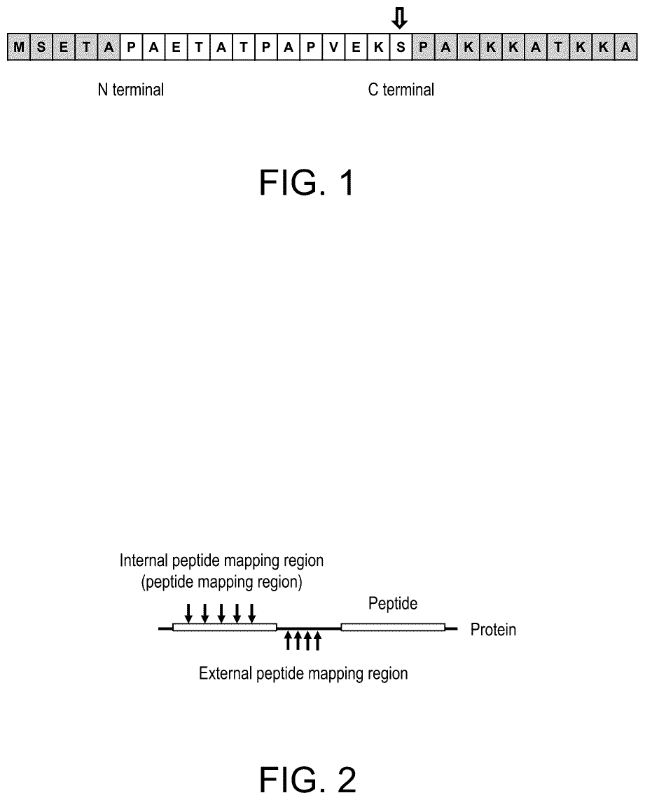

FIG. 1 is a schematic diagram of a conventional method for generating negative samples. According to the results of biological experiments, the yellow region is a peptide, and the cut point position is at the C terminal (taking FIG. 1 as an example, the cut point is S), and the C terminal is defined as a positive sample. The C terminal cut point position (the cut point position needs to be a positive sample) is excluded from the peptide (yellow region) as a possible negative sample. The method of finding negative samples from a single peptide may result in the negative samples found to contain the results of the N terminal cut point, while being mixed with negative samples with higher uncertainty. Therefore, in the invention, regions with high frequency and high density of peptide distribution are searched from the distribution relationship between peptides and protein sequences, and negative samples are defined therefrom, in order to find more representative negative samples.

The saturation defined in the literature (cleavage saturation, as shown in Method 1 below) screens out protein sequences with high saturation and searches for negative samples in the regions without peptide coverage in the protein sequence. However, this screening method only takes into account the aspect of the cut point, ignoring the impact of the quantity and the coverage of peptides, thus further making it more likely that the selection of negative samples contains noise. Therefore, in the invention, the characteristics of the source protein are described from a plurality of angles such as cut point and peptide distribution, and negative samples are further searched from regions without peptide coverage in the screened protein sequence, in order to find more representative negative samples.

$$Saturation = \frac{N}{{N}_{total}} \qquad formula 1$$

N: quantity of cut points with two or more coverage ranges
Nₜₒₜₐₗ: quantity of non-repeated C terminal or N terminal cut points

Based on the above, in the invention, the internal peptide mapping regions and the external peptide mapping regions are defined by describing the relationship between peptides and protein sequences in a data-driven manner (as shown in FIG. 2, FIG. 2 is a schematic diagram of defining the internal peptide mapping regions and the external peptide mapping regions), and negative samples are found from these representative regions.

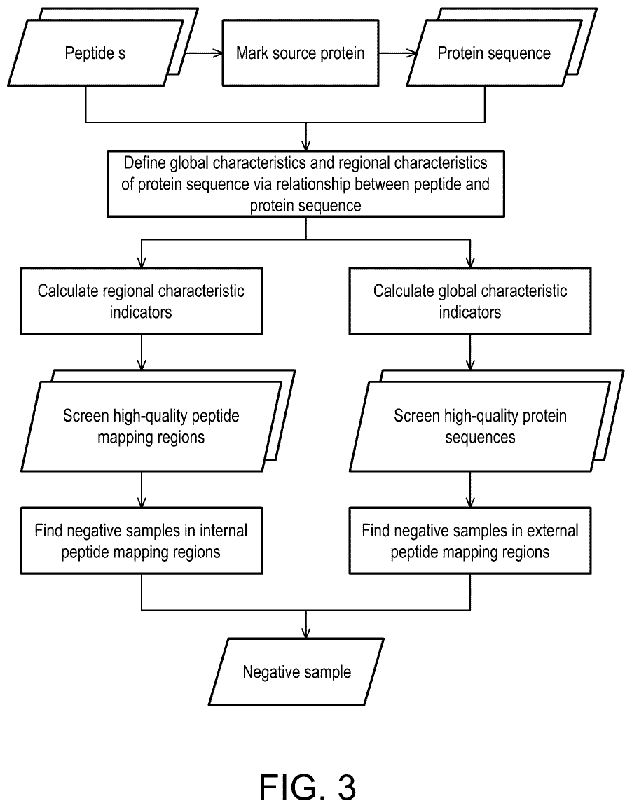

FIG. 3 is a schematic diagram of a method for generating negative samples based on the relationship between peptides and source proteins according to an embodiment of the invention.

Referring to FIG. 3, the invention provides a method for generating negative samples based on the relationship between peptides and source proteins, mainly involving marking a source protein (known), defining the global and regional characteristics of the protein sequence via the relationship between a peptide and a protein sequence, and then calculating protein sequence characteristic indicators, and screening out high-quality sequences based on the indicators. In the present embodiment, the source protein is marked by using the gene name and accession name corresponding to the source protein sequence (UniProt or SwissProt) in the database. High quality may be described as: after screening by characteristic indicators, protein sequences with high credibility and relatively clear functional and structural information are retained. According to the screening conditions here, negative samples are expected to be amino acid sites that are rarely cleaved or never cleaved.

In the present embodiment, the method for generating negative samples based on the relationship between peptides and source proteins of the invention includes the following steps. A source protein is marked, and global characteristics and regional characteristics of a protein sequence are defined via a relationship between a peptide and the protein sequence. Next, regional characteristic indicators and global characteristic indicators are calculated. Next, high-quality peptide mapping regions are screened out and negative samples are found in internal peptide mapping regions using the regional characteristic indicators, and high-quality protein sequences are screened out and negative samples are found in external peptide mapping regions using the global characteristic indicators.

The invention takes into account the distribution of cut points and the distribution of peptides in the source protein to quantify the characteristics of the protein. The global characteristic indicators include cut point saturation, peptide coverage, and peptide density. By sorting the three protein characteristic sequence indicators from large to small: peptide density, peptide coverage, and cut point saturation, the confidence level of the regions in the protein sequence not covered by peptides may be quantified as negative samples.

The cut point saturation is used to measure the consistency of the cut points of this protein sequence. The higher the cut point saturation, the more consistent the cut points are, meaning that the confidence level of the region without peptide coverage in this protein sequence as a negative sample candidate is higher; conversely, a low cut point saturation means that the cut points on this protein sequence are relatively inconsistent, meaning that the confidence level of the region without peptide coverage in this protein sequence as a negative sample candidate is reduced.

Peptide coverage is used to measure the breadth of peptide coverage of protein sequences. A high peptide coverage means that the protein sequence is readily cleaved by protease, and the confidence level of the region without peptide coverage in the protein sequence as a negative sample is increased, and vice versa.

Peptide density is used to measure the depth of peptide coverage of protein sequences. A high peptide density means that the protein sequence is readily cleaved by protease, and the confidence level of the region without peptide coverage in the protein sequence as a negative sample is increased, and vice versa.

In the present embodiment, the regional characteristic indicators are calculated in units of peptide mapping regions. The regional characteristic indicators include defining high-frequency peptide mapping regions and calculating and sorting peptide mapping region quantitative indicators.

Defining the high-frequency peptide mapping regions includes selecting amino acid sites for which coverage times are greater than the median of coverage times of the entire amino acid site as qualified amino acid sites, and continuous amino acid sites form a peptide mapping region.

The peptide mapping region quantitative indicators are calculated and sorted by calculating peptide density, peptide coverage, and cut point saturation in units of peptide mapping regions. Similarly, the higher the peptide density, the peptide coverage, and the cut point saturation values, the higher the confidence level of the negative samples in this peptide mapping region. The peptide density, the peptide coverage, and the cut point saturation are sorted from large to small to quantify the confidence level of the region without peptide coverage in the protein sequence as a negative sample.

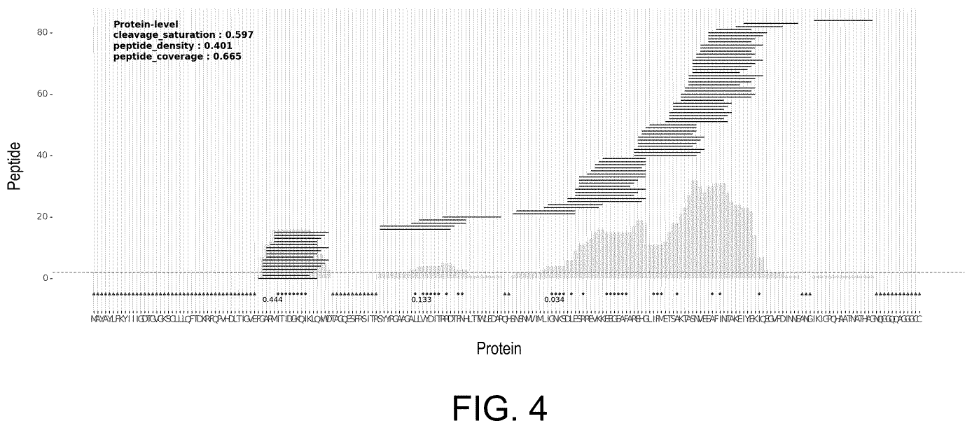

FIG. 4 is a schematic diagram of finding negative samples in a defined region according to an embodiment of the invention. In the present embodiment, high-quality peptide mapping regions are screened out and negative samples (such as green points in FIG. 4) are found in the internal peptide mapping regions using the regional characteristic indicators. Using global characteristic indicators, high-quality protein sequences are screened out, and negative samples (such as the brown dots in FIG. 4) are found in the external peptide mapping regions.

The following is a comparison of different processes for generating negative samples via an experimental protease cleavage model.

Protease cleavage model training process:

(1) Data preparation: negative sample sites are generated via the process of the invention using known peptide data, and the peptide data may include IEDB. A dataset of positive and negative sample sequences is formed from these site opening regions.

(2) Dataset segmentation: the dataset with a 1:1 ratio of positive and negative samples is segmented into a training dataset, a validation dataset, and a test dataset in a ratio of 8:1:1.

(3) Model building

I. Feature engineering: one-hot encoding features and physical and chemical characteristic features of the sequence are established respectively.

II. Model establishment: a sequential neural network model and a physical and chemical characteristic neural network model are established respectively, and the prediction probabilities of the two models (taking the average) are integrated as the final cut point prediction probability.

(4) Model prediction and evaluation: the model performance is tested on the test dataset and the model is measured via model evaluation indicators.

From Table 1 below, it may be seen that the method for generating negative samples via the process of the invention has better model performance.

TABLE 1

| | | | | NPV | | | | | | |
|---|---|---|---|---|---|---|---|---|---|---|
| | | | PPV | (Recall | | | | | | |
| Workflow | Sensitivity | Specificity | (Precision) | rate) | Accuracy | TP | FP | TN | FN | AUC |
| pepsickle | 0.65 | 0.6 | 0.617 | 0.63 | 0.62 | 14950 | 9265 | 13806 | 8121 | 0.67 |
| decoy | | | | | | | | | | |
| generation | | | | | | | | | | |
| Process of | 0.69 | 0.62 | 0.644 | 0.67 | 0.65 | 15881 | 8790 | 14281 | 7190 | 0.71 |
| invention | | | | | | | | | | |
| pepCleave | 0.69 | 0.61 | 0.638 | 0.66 | 0.65 | 15998 | 9096 | 13975 | 7073 | 0.71 |
| decoy | | | | | | | | | | |
| generation | | | | | | | | | | |

Based on the above, the invention provides a method for generating negative samples based on the relationship between peptides and source proteins. From the distribution relationship between peptides and protein sequences, regions with high frequency and high density of peptide distribution are found, and negative samples are defined therefrom. The characteristics of the source protein are described from a plurality of angles such as the cut point and peptide distribution. Further, negative samples are found from regions without peptide coverage in the screened protein sequence, in order to find more representative negative samples. Therefore, the performance of the protease cleavage prediction model may be enhanced to further optimize the design of peptide linkers with better model performance.

What is claimed is:

1. A method for generating negative samples based on a relationship between peptides and source proteins, comprising:
marking a source protein;
defining global characteristics and regional characteristics of a protein sequence via a relationship between a peptide and the protein sequence;
calculating regional characteristic indicators and global characteristic indicators; and
screening out peptide mapping regions and finding negative samples in internal peptide mapping regions using the regional characteristic indicators, and screening out protein sequences and finding negative samples in external peptide mapping regions using the global characteristic indicators.

2. The method for generating the negative samples based on the relationship between the peptides and the source proteins of claim 1, wherein the global characteristic indicators comprise a cut point saturation, a peptide coverage, and a peptide density.

3. The method for generating the negative samples based on the relationship between the peptides and the source proteins of claim 2, wherein the peptide density, the peptide coverage, and the cut point saturation are sorted from large to small to quantify a confidence level of a region without peptide coverage in the protein sequence as a negative sample.

4. The method for generating the negative samples based on the relationship between the peptides and the source proteins of claim 1, wherein the regional characteristic indicators comprise defining high-frequency peptide mapping regions and calculating and sorting peptide mapping region quantitative indicators.

5. The method for generating the negative samples based on the relationship between the peptides and the source proteins of claim 4, wherein defining the high-frequency peptide mapping regions comprises selecting amino acid sites for which coverage times are greater than a median of coverage times of an entire amino acid site as qualified amino acid sites, and continuous amino acid sites form a peptide mapping region.

6. The method for generating the negative samples based on the relationship between the peptides and the source proteins of claim 5, wherein the peptide mapping region quantitative indicators are calculated and sorted by calculating a peptide density, a peptide coverage, and a cut point saturation in units of peptide mapping regions.

7. The method for generating the negative samples based on the relationship between the peptides and the source proteins of claim 6, wherein the peptide density, the peptide coverage, and the cut point saturation are sorted from large to small to quantify a confidence level of a region without peptide coverage in the protein sequence as a negative sample.
